(19) United States
(12) Patent Application Publication
(10) Pub. No.: US 2025/0095844 A1
(43) Pub. Date: Mar. 20, 2025

(54) DETECTING BIOMECHANICAL IMPAIRMENT USING WEARABLE DEVICES

(71) Applicant: Apple Inc., Cupertino, CA (US)

(72) Inventor: Joseph Vedadi, Los Angeles, CA (US)

(21) Appl. No.: 18/889,262

(22) Filed: Sep. 18, 2024

Related U.S. Application Data
(60) Provisional application No. 63/539,562, filed on Sep. 20, 2023.

(51) Int. Cl. G16H 40/67; A61B 5/00; A61B 5/0205; A61B 5/11

(52) U.S. Cl. CPC G16H 40/67; A61B 5/0205; A61B 5/7267; A61B 5/746; A61B 5/112; A61B 5/6817

(57) ABSTRACT

Embodiments are disclosed for detecting biomechanical impairment using wearable devices. An example method comprises: obtaining a first set of sensor data, a location of the wearable device and a timestamp; determining a first set of fitness metrics based on the sensor data; predicting a baseline health profile based on the first set of fitness metrics; storing the baseline health profile, location and timestamp; at a second time after the timestamp: detecting that the wearable device is at the location; obtaining a second set of sensor data from the sensors of the wearable device; determining a second set of fitness metrics based on the second set of sensor data; predicting a current health profile for the user based on the second set of fitness metrics; comparing the current health profile with the baseline health profile; and responsive to a result of the comparing, performing an action.

### CROSS-REFERENCE TO RELATED APPLICATION

This application claims priority to U.S. Provisional Patent Application No. 63/539,562, filed Sep. 20, 2023, the entire contents of which are incorporated herein by reference.

### TECHNICAL FIELD

This disclosure relates generally to health monitoring.

### BACKGROUND

Individuals move their bodies and limbs in highly unique and repeatable patterns based on, for example, pelvic rotation, pelvic tilt, knee and hip flexion, knee and ankle interaction, and lateral pelvic displacement. Changes to these determinants can indicate health issues, such as injury or disease. Changes due to a progressive disease or aging occur slowly over many months or years, making such changes difficult for the individual or their doctor to observe during semi-annual or annual checkups. It is desirable, however, to detect such biomechanical aging or disease processes over the long term before they are observable in a clinical setting, so that preventative treatments can be prescribed and evaluated for effectiveness by the individual or doctor before they develop into more serious health problems.

### SUMMARY

Embodiments are disclosed for detecting biomechanical impairment using wearable devices. In some embodiments, a method comprises: obtaining a first set of sensor data, a location of the wearable device and a timestamp; determining a first set of fitness metrics based on the sensor data; predicting a baseline health profile based on the first set of fitness metrics; storing the baseline health profile, location and timestamp; at a second time after the timestamp: detecting that the wearable device is at the location; obtaining a second set of sensor data from the sensors of the wearable device; determining a second set of fitness metrics based on the second set of sensor data; predicting a current health profile for the user based on the second set of fitness metrics; comparing the current health profile with the baseline health profile; and responsive to a result of the comparing, performing an action.

In some embodiments, the sensors include inertial sensors, a heart rate sensor and at least one in-ear device.

In some embodiments, predicting the baseline and current health profiles include providing the first and second sets of fitness metrics as inputs to a machine learning model that is trained to predict an overall fitness score for the user.

In some embodiments, the machine learning model is trained using health profiles of other individuals at the location that have a similar health profile as the user.

In some embodiments, the machine learning model is trained on augmented historical health profiles of the user.

In some embodiments, the first and second sets of fitness metrics include at least one of gait analysis, walking steadiness or cardiorespiratory fitness.

In some embodiments, the action includes sending an alert notification to the user.

Other embodiments are directed to an apparatus, system and computer-readable medium.

Particular embodiments described herein provide one or more of the following advantages. This disclosed system and method uses machine learning to generate a baseline user health profile over time (e.g., one month) based on sensor measurements output by various sensors of a wearable devices (e.g., smartphones, smartwatches, fitness devices, earbuds, head mounted devices, chest mounted devices). The user baseline health profile can be associated with a particular geographic location and/or time. The detection of sudden or long-term changes to the user's baseline health profile can trigger notification to the user (e.g., visual, audio or haptic notification) indicating that the user's current health profile deviated (e.g., deteriorated) from the user's baseline health profile, and the particular metrics that caused the deviation (e.g., uneven gait, change in walking speed, breathing pattern, bone vibration, inner sound, Bruxism, joint cracking).

### BRIEF DESCRIPTION OF THE DRAWINGS

FIG. 1 is a block diagram of system for detecting biomechanical impairment using wearable devices, according to one or more embodiments.

FIG. 2 is a flow diagram of a process of detecting biomechanical impairment using wearable devices, according to one or more embodiments.

FIG. 3 is a block diagram of a device architecture for implementing the features and processes described in reference to FIGS. 1-2.

### DETAILED DESCRIPTION

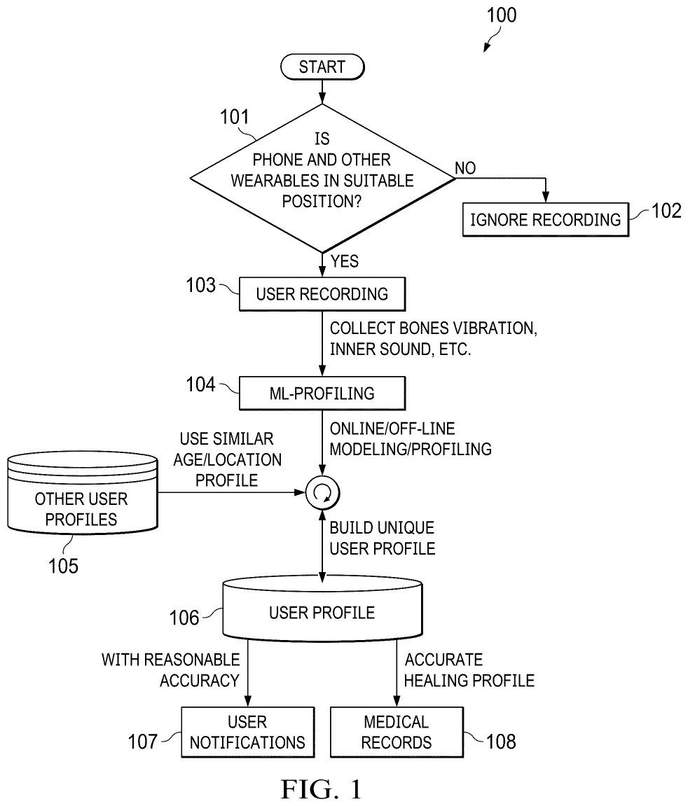

FIG. 1 is a flow diagram of process 100 for detecting biomechanical impairment using wearable devices, according to one or more embodiments. Process 100 includes determining if a wearable device is available for recording (101). For example, a wearable device could be a smartphone, a smartwatch, earbuds, chest mounted device, head mounted device (HMD), etc. The wearable device is available for recording if it is powered on and positioned on the user's body such that sensor measurements can be made by one or more sensors of the wearable device. For example, earbuds are available for recording if powered on and inserted in the user's ear. A microphone in the earbud captures the user's inner body sound. Another example is a heart rate sensor embedded in a smartwatch attached to the user's wrist that measures the user's heart rate. A wearable device that is unavailable for recording would be, for example, a wearable device that is not on the user's body or is out of power. In some embodiments, a machine learning model is trained to detect if a wearable device is on the user, such as detecting if a smartphone is in a pants pocket.

If at least one wearable device is available for recording, sensor measurements are recorded 103 on the wearable device and stored on the wearable device and/or uploaded to a network storage device, or to storage on a paired device (e.g., a smartwatch paired with a smartphone). Otherwise, sensor measurements are not recorded 202. Some examples of sensor measurements include but are not limited to bone vibration, inner body sounds (e.g., joints cracking, breathing, teeth grinding, etc.), heart rate, user body motion (e.g., acceleration, rotation rate, step frequency step), respiratory noises captured by an external microphone of the wearable device (e.g., heavy breathing, holding breath, wheezing), electroencephalogram (EEG) signals, electrocardiogram (ECG) signals, temperature, VO2 max and blood volume pulse (BVP) obtained from a photoplethysmography (PPG) sensor, etc. From the motion sensor data, various other measurements or metrics can be derived including but not limited to the user's walking pattern (e.g., falling and catching), walking speed, gait balance, etc.

The sensor data described above can be captured simultaneously at the same data rate or at different data rates. In addition to the sensor data, the user's location where the sensor data was obtained, and a time stamped indicating the date and time the sensor data was obtained are also recorded and stored with the sensor data. The sensor data can be continuously captured/measured, periodically captured/measured and/or captured/measured in response to a trigger event.

In some embodiments, the stored sensor data are input into a machine learning model 104 that is trained or configured to predict a health profile for the user. Some examples of machine learning models include but are not limited to deep learning networks (e.g., convolutional neural network (CNN)), k-nearest neighbor clustering (KNN), naïve Bayes, regression analysis (linear and logistic), decision trees, random forest and support vector machines. In some embodiments, feature vectors are formed from the stored sensor data. The feature vectors are input into a machine learning model that has been trained to predict a user health profile 106. In some embodiments, the machine learning model is trained using health profiles 105 of other individuals at the location that have a similar health profile as the user. In some embodiments, the machine learning model is trained on augmented historical health profiles of the user to increase the amount of training data.

In some embodiments, the machine learning model predicts a baseline health profile 106. This prediction would ideally occur when the user is healthy and free of injuries. The baseline health profile 106 can include one or more fitness scores or metrics that indicate the fitness of the user. For example, a machine learning model can predict an overall health score for the user based on gait analysis of the user, as described in U.S. Patent Publication No. 2021/0393166, for “Monitoring User Health Using Gait Analysis,” published Dec. 23, 2021, which patent publication is incorporated by reference herein in its entirety. In some embodiments, the machine learning model can predict the cardiorespiratory fitness of a user as described in co-pending U.S. patent application Ser. No. 17/985,098, for “Identifying Poor Cardiorespiratory Fitness Using Sensors of Wearable devices, filed Nov. 11, 2022, which patent application is incorporated by reference herein in its entirety. In some embodiments, the machine learning model can predict walking steadiness, as described in U.S. Patent Publication No. 2023/0112071 A1, for “Assessing Fall Risk of Mobile Device User,” published Apr. 13, 2023, which patent publication is incorporated by reference herein in its entirety.

In some embodiments, a trained machine learning model is used to determine if the wearable device is on a dominant or regressive leg of the user (e.g., a smartphone in the right or left front pants pocket) to determine if the user is limping, which could be an indicator of poorly fitting shoes, strains, sprains, or an object such as a splinter stuck in the foot, misbalance, bone fracture, etc.

Accordingly, the scores or metrics computed by the above-referenced processes can comprise the user's baseline health profile 106. If these scores or metrics change from the baseline by a specified amount, a notification 107 (e.g., push notification, text message, display) can be sent to the user's wearable device or other device to alert the user of the change and indicate which score/metric caused the change. In some embodiments, with the user's permission the user's health profile 106 can be included into the user's medical records 108 where it can be accessed by health professionals.

For example, in a first month a first set of sensor data is obtained by a user's smartwatch and earbuds while the user is wearing the devices. Sensors in the smartwatch measure motion data (e.g., shaking, vibration) and heart rate. Microphones on the smartwatch capture the user's breathing. A microphone inside the earbud captures inner body sounds from top of the neck. The sensor data is used to derive additional fitness metrics that indicate the user's gait, walking steadiness and cardiorespiratory fitness, as described above. The metrics are input into a machine learning model which predicts an overall fitness score for the user. The system searches for the user's health profile stored on the wearable device, companion device or on a network. Since this is the first instance, the system creates a new user health profile and labels it as a baseline health profile. The overall fitness score is included in the baseline health profile stored on the smartwatch and/or on the companion device and/or network storage.

Additionally, a global navigation satellite system (GNSS) receiver (e.g., a global positioning system (GPS) receiver) on the smartwatch determines the location of the smartwatch. The location, and a timestamp are also stored in the baseline health profile. Also, an activity classifier on the wearable device provides an indication of the activity the user was engaged in when the sensor data was obtained (e.g., walking, jogging, cycling, driving).

The wearable device monitors the user's location (e.g., from GPS output) and time to determine when the user is at the same location where the baseline user profile was created and a threshold amount of time after the baseline user health profile was created (e.g., 1 month later). In some embodiments, a geofence is set around the location which triggers a second set of sensor data to be obtained when crossed by the user.

The second set of sensor data is obtained by the user's smartwatch and earbuds. Again, the second set of sensor data includes motion data, heart rate breathing and inner body sounds. The second set of sensor data is input in the machine learning model which predicts a second overall fitness score. The second overall fitness score is compared to stored first overall fitness score stored in the baseline user health profile. If the second overall fitness score is lower than the baseline overall fitness score by a specified amount, then an action is performed, such as sending an alert message to the user.

The alert message can be displayed on the smartwatch or companion device, and/or a push notification or text message can be sent to the wearable device or other device. The alert message can inform the user that their overall fitness has deteriorated since the previous month. The current health profile becomes a new baseline health profile that is stored so that it can be compared with new health profile for the user that is computed in the following month, a so forth.

As described above, an activity classifier on the wearable device can determine the activity that the user is engaged in at the time sensor data is obtained. In some embodiments, the overall fitness metric is not computed if the activity the user is currently engaged is different than the activity the user was engaged in when the baseline health profile was created in the previous month. An example activity classifier is described in U.S. Pat. No. 8,892,391, for “Activity Detection,” Issued Nov. 18, 2014, which patent is incorporated by reference herein in its entirety.

In some embodiments, the individual metrics that contributed to the overall fitness score (e.g., a walking mobility metric, cardiorespiratory metric) can be provided in the alert message so that the user can see which specific aspects of their health profile caused the deterioration in the overall fitness score from the baseline overall fitness score of the preceding month. In this example, a gate analysis metric indicates that the user was falling and catching themselves and the cardiorespiratory metric indicated a longer than usual recovery time after performing a physical activity. This information can encourage the user to focus on their health and provide useful health information that the user can share with their doctor.

In some embodiments, the user can set the time interval between sensor measurements (e.g., 1 week, 1 month, 3, months, 6 months, 1 year, etc.) in a settings pane of the wearable device or through other suitable input. The user can also select a preference for receiving alert messages (e.g., push notification, text message, etc.) and a desired format for a health profile summary (e.g., determine what metrics are shown).

In some embodiments, the foregoing is part of a health-related mobile application. The user can request through the application a real-time analysis to check if, e.g., a new pair of shoes are good fit. For example, bad fitting shoes could impact the user's walking gait. The user could also request that the application track their spinal health and physical improvement and posture in response to treatments. For example, track chiropractor adjustment progress or walking balance. In some embodiments, feedback could be used to implement a weight loss/gain program.

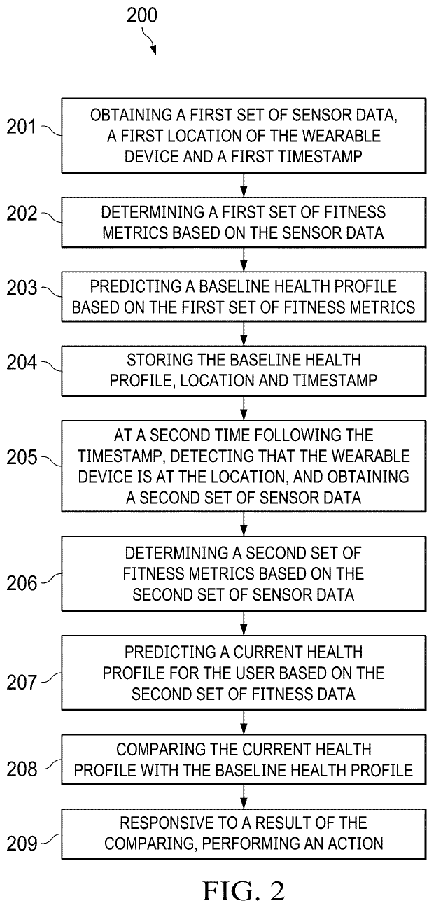

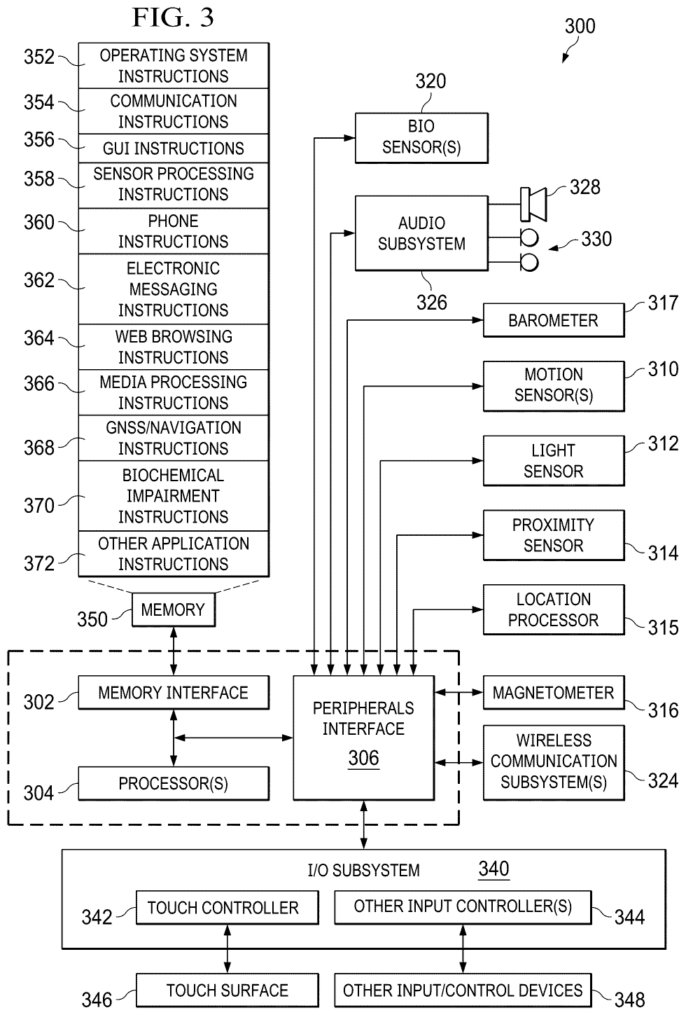

FIG. 2 is a flow diagram of a process 200 of detecting biomechanical impairment using wearable devices, according to one or more embodiments. Process 200 can be implemented in, e.g., device architecture 300 described in reference to FIG. 3.

Process 200 includes, at a first time, obtaining a first set of sensor data from sensors of a wearable device, a first location of the wearable device and a timestamp (201); determining a first set of fitness metrics based on the sensor data (202); predicting a baseline health profile for a user of the wearable device based on the first set of fitness metrics (203); storing the baseline health profile, the location and the timestamp (204); at a second time after the timestamp: detecting that the wearable device is at the location, obtaining a second set of sensor data from the sensors of the wearable device (205); determining a second set of fitness metrics based on the second set of sensor data (206); predicting a current health profile for the user of the wearable device based on the second set of fitness data (207); comparing the current health profile with the baseline health profile (208); and responsive to a result of the comparing, performing an action (209). Each of these steps were previously described in reference to FIG. 1.

In some embodiments, the sensors include inertial sensors, a heart rate sensor and at least one in-ear device that can measure, e.g., inner body sounds at the top of the neck (e.g., cracking). In some embodiments, predicting the baseline and current health profiles include providing the first and second sets of fitness metrics as inputs to a machine learning model that is trained to predict an overall fitness score for the user. For example, the machine learning model can be a deep learning neural network trained on health profiles of other individuals of the same age and similar health profile. In some embodiments, the first and second sets of fitness metrics include at least one of gait analysis, walking steadiness or cardiorespiratory fitness. In some embodiments, the action taken includes sending an alert notification to the user, which can be in the form of a push notification, text message, visual display, haptic feedback, audio feedback, etc.

FIG. 3 is a block diagram of a device architecture 300 for implementing the features and processes described in reference to FIGS. 1-2. Architecture 300 can include memory interface 302, one or more hardware data processors, image processors and/or processors 304 and peripherals interface 306. Memory interface 302, one or more processors 304 and/or peripherals interface 306 can be separate components or can be integrated in one or more integrated circuits. System architecture 300 can be included in any suitable electronic device for crash detection, including but not limited to: a smartwatch, smartphone, fitness band and any other device that can be attached, worn, or held by a user.

Sensors, devices, and subsystems can be coupled to peripherals interface 306 to provide multiple functionalities. For example, one or more motion sensors 310, light sensor 312 and proximity sensor 314 can be coupled to peripherals interface 306 to facilitate motion sensing (e.g., acceleration, rotation rates), lighting and proximity functions of the wearable device. Location processor 315 can be connected to peripherals interface 306 to provide geo-positioning. In some implementations, location processor 315 can be a GNSS receiver, such as the Global Positioning System (GPS) receiver. Electronic magnetometer 316 (e.g., an integrated circuit chip) can also be connected to peripherals interface 306 to provide data that can be used to determine the direction of magnetic North. Electronic magnetometer 316 can provide data to an electronic compass application. Motion sensor(s) 310 can include one or more accelerometers and/or gyros configured to determine change of speed and direction of movement. Barometer 317 can be configured to measure atmospheric pressure (e.g., pressure change inside a vehicle). Bio signal sensor 320 can be one or more of a PPG sensor, an electroencephalogram (EEG) sensor, an electrocardiogram (ECG) sensor, an electromyogram (EMG) sensor, a mechanomyogram (MMG) sensor (e.g., piezo resistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 324, which can include radio frequency (RF) receivers and transmitters (or transceivers) and/or optical (e.g., infrared) receivers and transmitters. The specific design and implementation of the communication subsystem 324 can depend on the communication network(s) over which a mobile device is intended to operate. For example, architecture 300 can include communication subsystems 324 designed to operate over a GSM network, a GPRS network, an EDGE network, a WiFi™ network and a Bluetooth™ network. In particular, the wireless communication subsystems 324 can include hosting protocols, such that the crash device can be configured as a base station for other wireless devices.

Audio subsystem 326 can be coupled to a speaker 328 and a microphone 330 to facilitate voice-enabled functions, such as voice recognition, voice replication, digital recording and telephony functions. Audio subsystem 326 can be configured to receive voice commands from the user.

I/O subsystem 340 can include touch surface controller 342 and/or other input controller(s) 344. Touch surface controller 342 can be coupled to a touch surface 346. Touch surface 346 and touch surface controller 342 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 346. Touch surface 346 can include, for example, a touch screen or the digital crown of a smart watch. I/O subsystem 340 can include a haptic engine or device for providing haptic feedback (e.g., vibration) in response to commands from processor 304. In an embodiment, touch surface 346 can be a pressure-sensitive surface.

Other input controller(s) 344 can be coupled to other input/control devices 348, such as one or more buttons, rocker switches, thumbwheel, infrared port, and USB port. The one or more buttons (not shown) can include an up/down button for volume control of speaker 328 and/or microphone 330. Touch surface 346 or other controllers 344 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 546; and a pressing of the button for a second duration that is longer than the first duration may turn power to the mobile device on or off. The user may be able to customize a functionality of one or more of the buttons. The touch surface 346 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 302 can be coupled to memory 350. Memory 350 can include high-speed random-access memory and/or non-volatile memory, such as one or more magnetic disk storage devices, one or more optical storage devices and/or flash memory (e.g., NAND, NOR). Memory 350 can store operating system 352, such as the iOS operating system developed by Apple Inc. of Cupertino, California. Operating system 352 may include instructions for handling basic system services and for performing hardware dependent tasks. In some implementations, operating system 352 can include a kernel (e.g., UNIX kernel).

Memory 350 may also store communication instructions 354 to facilitate communicating with one or more additional devices, one or more computers and/or one or more servers, such as, for example, instructions for implementing a software stack for wired or wireless communications with other devices. Memory 350 may include graphical user interface instructions 356 to facilitate graphic user interface processing; sensor processing instructions 358 to facilitate sensor-related processing and functions; phone instructions 360 to facilitate phone-related processes and functions; electronic messaging instructions 362 to facilitate electronic-messaging related processes and functions; web browsing instructions 364 to facilitate web browsing-related processes and functions; media processing instructions 366 to facilitate media processing-related processes and functions; GNSS/Location instructions 368 to facilitate generic GNSS and location-related processes and instructions; and instructions 370 that implement the processes described in reference to FIGS. 1-2. Memory 350 further includes other application instructions 372 including but not limited to instructions for applications that implement system 100 shown in FIG. 1.

Each of the above identified instructions and applications can correspond to a set of instructions for performing one or more functions described above. These instructions need not be implemented as separate software programs, procedures, or modules. Memory 350 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
at a first time:
obtaining, with at least one processor, a first set of sensor data from sensors of a wearable device, a location of the wearable device and a timestamp;
determining, with the at least on processor, a first set of fitness metrics based on the sensor data;
predicting, with the at least one processor, a baseline health profile for a user of the wearable device based on the first set of fitness metrics;
storing, with the at least one processor, the baseline health profile, the location and the timestamp;
at a second time after the timestamp:
detecting, with the at least one processor, that the wearable device is at the location;
obtaining, with at least one processor, a second set of sensor data from the sensors of the wearable device;
determining, with the at least on processor, a second set of fitness metrics based on the second set of sensor data;
predicting, with the at least one processor, a current health profile for the user of the wearable device based on the second set of fitness metrics;
comparing, with the at least one processor, the current health profile with the baseline health profile; and
responsive to a result of the comparing, performing an action.

2. The method of claim 1, wherein the sensors include inertial sensors, a heart rate sensor and at least one in-ear device.

3. The method of claim 1, wherein predicting the baseline and current health profiles include providing the first and second sets of fitness metrics as inputs to a machine learning model that is trained to predict an overall fitness score for the user.

4. The method of claim 3, wherein the machine learning model is trained using health profiles of other individuals at the location that have a similar health profile as the user.

5. The method of claim 3, wherein the machine learning model is trained on augmented historical health profiles of the user.

6. The method of claim 1, wherein the first and second sets of fitness metrics include at least one of gait analysis, walking steadiness or cardiorespiratory fitness.

7. The method of claim 1, wherein the action includes sending an alert notification to the user.

8. A system comprising:
at least one processor;
memory storing instructions that when executed by the at least one processor cause the at least one processor to perform operations comprising:
at a first time:
obtaining a first set of sensor data from sensors of a wearable device, a location of the wearable device and a timestamp;
determining a first set of fitness metrics based on the sensor data;
predicting a baseline health profile for a user of the wearable device based on the first set of fitness metrics;
storing the baseline health profile, the location and the timestamp;
at a second time after the timestamp:
detecting that the wearable device is at the location;
obtaining a second set of sensor data from the sensors of the wearable device;
determining a second set of fitness metrics based on the second set of sensor data;
predicting a current health profile for the user of the wearable device based on the second set of fitness metrics;
comparing the current health profile with the baseline health profile; and
responsive to a result of the comparing, performing an action.

9. The system of claim 8, wherein the sensors include inertial sensors, a heart rate sensor and at least one in-ear device.

10. The system of claim 8, wherein predicting the baseline and current health profiles include providing the first and second sets of fitness metrics as inputs to a machine learning model that is trained to predict an overall fitness score for the user.

11. The system of claim 10, wherein the machine learning model is trained using health profiles of other individuals at the location that have a similar health profile as the user.

12. The system of claim 10, wherein the machine learning model is trained on augmented historical health profiles of the user.

13. The system of claim 8, wherein the first and second sets of fitness metrics include at least one of gait analysis, walking steadiness or cardiorespiratory fitness.

14. The system of claim 8, wherein the action includes sending an alert notification to the user.
